(19) United States
(12) Patent Application Publication
(10) Pub. No.: US 2013/0131140 A1
(43) Pub. Date: May 23, 2013

(54) TREATMENT OF DIABETES AND DISORDERS ASSOCIATED WITH VISCERAL OBESITY WITH INHIBITORS OF HUMAN ARACHIDONATE 12 LIPOXYGENASE AND ARACHIDONATE 15-LIPOXYGENASE

(71) Applicants: Jerry L. Nadler, Norfolk, VA (US); David Taylor-Fishwick, Norfolk, VA (US); Swarup Chakrabarti, Norfolk, VA (US); Anca Dobrian, Norfolk, VA (US)

(72) Inventors: Jerry L. Nadler, Norfolk, VA (US); David Taylor-Fishwick, Norfolk, VA (US); Swarup Chakrabarti, Norfolk, VA (US); Anca Dobrian, Norfolk, VA (US)

(21) Appl. No.: 13/522,165

(22) PCT Filed: Jan. 14, 2011
(86) PCT No.: PCT/US11/21300

§ 371 Date: Feb. 11, 2013

Related U.S. Application Data
(60) Provisional application No. 61/295,078, filed on Jan. 14, 2010.
(60) Provisional application No. 61/306,129, filed on Feb. 19, 2010.
(60) Provisional application No. 61/326,888, filed on Apr. 22, 2010.

(51) Int. Cl. C12N 15/113; C12Q 1/02

(52) U.S. Cl. CPC C12N 15/1137; C12Q 1/025
USPC 514/44 A; 435/6.13; 435/6.12

(57) ABSTRACT

A basis for understanding the arachidonate 12-lipoxygenase pathway, as well as and methods and kits for inhibiting the arachidonate 12-lipoxygenase pathway for the treatment, reversal, reduction, modulation or prevention of disease states and conditions related to type 1 or type 2 diabetes, are disclosed. Also disclosed are inflammatory forms of ALOX12 and 15, which are selectively expressed in omental adipose tissue of obese humans. Inhibitors of ALOX12 and 15 can be used to treat, prevent, modulate or reduce complications associated with increased visceral obesity and inflammation, including type 2 diabetes. Also disclosed are methods for developing selective ALOX inhibitors for treating or reducing complications associated with increased visceral obesity and inflammation.

### CROSS-REFERENCE TO RELATED APPLICATION

This application claims the benefit of U.S. Provisional Patent Application No. 61/295,078, filed Jan. 14, 2010, U.S. Provisional Patent Application No. 61/306,129, filed Feb. 19, 2010, and U.S. Provisional Patent Application No. 61/326,888, filed Apr. 22, 2010, which are hereby incorporated by reference herein in their entirety.

### STATEMENT CONCERNING GOVERNMENT RIGHTS IN FEDERALLY-SPONSORED RESEARCH

This invention was made with government support under Grant Nos. R01DK055240 and P01 HL55798 awarded by the National Institutes of Health. The government has certain rights in this invention.

### FIELD OF THE INVENTION

This invention pertains to modulation of the human 12-lipoxygenase pathway, found in pancreatic islet cells, arachidonate LO-12, which is also known as platelet-type 12-LO, or ALOX12, particularly products of the pathway, 12(S)-HETE and 12-HPETE, which reduce insulin secretion and increase pancreatic islet cell death. In addition, this invention pertains to human 12-lipoxygenase and 15-lipoxygenase (12/15 ALOX) isoforms found in human adipose tissue and the inhibition of these isoforms to regulate several disease states associated with activity of these isoforms. The invention further pertains to assays that can identify agents that modulate the activity of the human 12-lipoxygenase and 15-lipoxygenase isoforms and to methods and compositions for treating type 1 and type 2 diabetes and complications arising from obesity.

### BACKGROUND

Obesity is associated with inflammation and insulin resistance, which promote the development of type 2 diabetes, as well as cardiovascular disease. Chronic exposure to high lipid levels triggers an inflammatory response that can damage the pancreas. The inflammatory mediators include cytokines, reactive oxygen species (ROS) and lipid factors that contribute to insulin resistance, pancreatic islet dysfunction, and the formation of atherosclerotic placques.

Lipoxygenases (LOs) are a family of iron-containing enzymes that catalyze the dioxygenation of polyunsaturated fatty acids in lipids. They are classified as 5-, 8-, 12-, and 15-LO according to the carbon atom of arachidonic acid at which the oxygen is inserted (1, 2). The 12-LO enzyme, but not 5-LO or 15-LO, is specifically expressed in pancreatic f3-cells (3). LO activity in human islets produces hydroxyeicosatetraenoic acids (HETEs). Little is known about the differential effect of the various HETEs that result from LO activity in human islets.

Type 1 diabetes is an autoimmune disorder associated with complete destruction of insulin producing β-cells. The cytokine-induced destruction of pancreatic β-cells seen in type 1 diabetes and islet graft rejection involves multiple intracellular signaling pathways that directly or indirectly lead to inflammatory damage or programmed cell death (6). Inflammation is also an important pathological process leading to β-cell dysfunction and death in type 2 diabetes (7).

The 12-lipoxygenase (12-LO) pathway is a link between inflammation, autoimmunity and (3-cell damage. Inflammatory cytokines rapidly activate 12-LO, and the cytokines acting on immune cells in pancreatic islets also can induce inflammatory genes and cytokine release.

Previous studies have been conducted with rodents, which have major differences from human islets (9). The role of 12-LO in type 1 diabetes development in mice has been demonstrated in global 12-LO knockout mice on the C57BL/6J background and congenic 12-LO null mice on the nonobese diabetic background (4,5). In these studies, 12-LO deletion led to a marked reduction of the rate of diabetes development. In β-cell lines such as β-TC3 cells, 12(S)-HETE added for a prolonged period also produced reduced insulin output (8). 12(S)-HETE-induced β-cell death in β-TC3 cells was possibly the result of the deregulation of the JNK and p38-MAPK kinase pathways in mouse cell lines (8).

Moreover, inflammation is increasingly recognized as an important contributing factor in diabetes mellitus. LOs produce active lipids that promote inflammatory damage by catalyzing the oxidation of linoleic and arachidonic acid. 12/15 lipoxygenases (12/15 LO) are specific isoforms that regulate the expression of pro-inflammatory cytokines and chemokines in different tissues. In vivo rodent studies have demonstrated that deletion of 12/15-LOs reduce inflammatory cytokine production and completely prevent insulin resistance in animals fed a Western diet (35,36). In vitro studies show that direct addition of 12/15-LO products to isolated adipocytes induces inflammatory cytokine expression and impairs insulin action (37). Studies using human macrophages demonstrate that overexpression of 12/15-LO stimulates the production of various chemokines and cytokines including IL-12a and increases T cell migration (38).

Adipose tissue inflammation plays a central role in obesity-related metabolic and cardiovascular complications. Obese individuals have at least six times the risk for having type 2 diabetes, leading to increases in cardiovascular morbidity and mortality. Multiple factors have been implicated in obesity-related metabolic and cardiovascular complications, including inflammatory cytokines such as TNFα, IL-6, IL-12 and IFNγ (40).

A basis for understanding lipoxygenase pathway for the development of inhibitors of this pathway for the treatment, reversal, reduction, modulation or prevention of disease states and conditions related to type 1 or type 2 diabetes or obesity is needed in view of the foregoing.

### SUMMARY

The invention is related to the determination as disclosed herein that inhibiting the human 12-LO pathway in pancreatic islet cells can be used to treat, reverse, modulate or prevent reduced insulin secretion, insulin resistance, and increased cell death in a human patient with or at risk of developing type 1 or type 2 diabetes, or a human patient receiving an islet graft, by modulating 12-LO products, 12(S)-HETE and 12-HPETE, that increase islet cell death in β-cells in the pancreas and reduce insulin secretion.

Accordingly, this invention can be used to identify and screen for inhibitors that reduce 12-LO activity, particularly those inhibitors that inhibit or modulate 12(S)-HETE and 12-HPETE, to reduce islet cell death and improve insulin secretion in β-cells in the pancreas. The disclosed methods, kits, and compositions can be used to inhibit the 12-LO pathway in a patient suffering from, or at risk of developing, type 1 or type 2 diabetes.

12/15 lipoxygenases (12/15 ALOX) play a major role in generating inflammatory mediators that lead to insulin resistance and vascular disease in rodent models of obesity. Adipose tissue (AT) inflammation is a determinant of insulin resistance in obese animal models.

ALOX 15a, it has been discovered, is selectively expressed only in omental (om) adipose tissue (e.g., the fat covered by the peritoneum, below the subcutaneous tissue), while ALOX 15b and 12 are present in both om and subcutaneous (sc) adipose tissue in diabetic and non-diabetic subjects. Increased expression of inflammatory mediators and activated macrophage and T cell infiltration occurs in omental adipose tissue of diabetic obese individuals compared to omental adipose tissue from non-diabetics. Increases of ALOX isoforms were found in om adipose tissue of diabetic versus non-diabetic subjects. Moreover, increased expression of inflammatory mediators and activated macrophage and T cell infiltration were observed in the adipose tissue of diabetic obese subjects. Inhibiting the 12/15-LO pathway, particularly ALOX12, ALOX 15a and ALOX 15b, is a useful method for treating insulin resistance and related vascular complications of inflammation and obesity.

Disclosed herein are methods, kits, and compositions for the treatment, reversal, modulation or prevention of insulin resistance and related vascular, heart, renal, hepatic, ocular, and other complications of inflammation and obesity in a patient, including type 2 diabetes. For example, the disclosed methods, kits, and compositions can be used to inhibit or modulate the 12-LO and/or 15-LO pathway in a patient suffering from, or at risk of developing, insulin resistance or related vascular complications of inflammation and obesity. This can also be accomplished by administering to a patient a compound or compounds that reduce or inhibit 12-LO and/or 15-LO activity to treat or prevent the complications associated with increased visceral obesity and inflammation.

### BRIEF DESCRIPTION OF THE DRAWINGS

The foregoing and other objects of the present inventions, the various features thereof, as well as the inventions themselves, may be more fully understood from the following description, when read together with the accompanying figures, in which:

FIG. 1 is a schematic, which shows the effects of inflammation and free fatty acids (FFAs) on inflammatory mediators and decreased islet function.

FIG. 2 is a set of graphs, which show that inflammatory cytokines induce 12-LO gene expression in human islets.

FIG. 3A is an image of a Western blot, which shows the effects of 12(S)-HETE and 12-LO blockade on pp38-MAPK protein activity.

FIG. 3B is a graph that depicts quantified protein levels for each protein in panel A, normalized to an actin control.

FIG. 4 is a graph, which shows inflammatory gene expression profiling in human islets.

FIG. 5 is a graph, which shows the increase in ALOX12 expression in human islets of five donors as a result of proinflammatory cytokine stimulation.

FIG. 6 is a graph, which shows the increase in ALOX12 expression in human islets of five donors as a result of proinflammatory cytokine stimulation.

FIG. 7 is a graph, which shows real-time expression of 12-LO and 5-LO (inset) when human islets were stimulated with cytokines (TNFα, IL-1β, INFγ).

FIG. 8 is an image, which shows direct induction of apoptosis in human islets stimulated with 12-HETE.

FIGS. 9A and 9B graphs, which show dose and time dependent changes in gene expression for interleukin-12 proteins (IL-12p35 and IL-12p40), and interferon-gamma (IFNγ) in islets from two donors (A and B) treated with 1 nM and 100 nM 12-HETE.

FIG. 10A is a schematic of molecular interactions relevant for proinflammatory cytokine (TNFα, IL-1β, INFγ) induction of pathways coincident with stimulation of islet apoptosis.

FIGS. 10B and 10C are images of islet viability determined using a stain for apoptosis (YO-PRO-1, green) and cell death (propidium iodide, red) in proinflammatory cytokine treated islets (B) and proinflammatory cytokine treated islets with the inhibitor (12-LO-I) of ALOX12 activity (C).

FIG. 11 is a graph, which shows a 100 fold up regulation of the key receptor of IL-12 (Beta 2 isoform) selectively in islets from a type 1 diabetic.

FIG. 12 is a graph, which shows HETE effects on human islet insulin secretion after treatment with several different LO products (HETEs) in 3.3 mM or 18 mM glucose at either 1 nM (A) or 100 nM (B) concentrations.

FIGS. 13A and 13B are graphs that show HETE effects on human islet insulin secretion after treatment with several different HETEs for 4 hours in 11 mM glucose at either 1 nM (A) or 100 nM (B) concentrations.

FIGS. 14A and 14B are graphs, which show that (A) the effects of 12(S)-HETE (12sH) on metabolic activity and insulin secretion are partially reversed by the small molecular anti-inflammatory compound lisofylline (LSF) as measured by MTT assay, and that (B) 12(S)-HETE-induced reduction in insulin secretion is reversed by LSF treatment.

FIGS. 15A and 15B are images of Western blots showing that HETEs induce cell death in human islets, in which (A) human islets (top) cultured overnight in untreated conditions, show relatively little cell death measured by PI fluorescence intensity (bottom), and that (B), overnight treatment with 12(S)-HETE which causes a significant increase in cell death at 100 nM.

FIG. 15C is a graph, which shows dose-dependent cell death response to 12(S)-HETE.

FIG. 16 is an image of a Western blot showing the effects of siRNA knockdown of 12-LO on pp38-MAPK, p38-MAPK (p38), and phosphorylated JNK expression (ppJNK1 and -2) protein levels in mouse islets compared with untreated controls (vehicle) or mice injected with missense siRNA (si-Control).

FIG. 17A represents a paired analysis of lipoxygenases and cytokines in sc vs. om tissue from obese subjects (n=12) by real-time PCR in which data was expressed as 1/Ct normalized to housekeeping gene.

FIG. 17B shows graphs that represent relative gene expression of lipoxygenases and cytokines in adipocytes and SVF was expressed using the total fat as a reference in om and sc depots (n=4).

FIG. 17C is an image which illustrates ALOX15a strongly and selectively expressed in om vasculature.

FIGS. 18A and 18B are sections of human islets stained for ALOX-12.

### DETAILED DESCRIPTION

1. Inhibition of the 12-LO Pathway in Human Pancreatic Islets

The 12-LO pathway is present in the β-cells of the pancreas. Activation of the 12-LO pathway leads to the formation of leukotrienes and also catalyzes the conversion of arachidonic acid to HETE by glutathione peroxidase. The products of the 12-LO pathway include 12-hydroxyeicosatetraenoic acid (12-(S)HETE), 12(R)-hydroxyeicosatetraenoic acid (12-(R)HETE) and 12-hydroperoxyeicosatetraenoic acid (12-HPETE).

The role of 12(S)-HETE, 12(R)-HETE, and 12-HPETE and 15-hydroxyeicosatetraenoic acid (15-HETE), from the 15-LO pathway, were examined in insulin secretion, β-cell metabolism, and cell viability in human islets. The 12-LO pathway activity leads to progressive decline in islet β-cell function, cell mass, and eventually, cell death. Potential mechanisms of 12-LO product activation and possible in vivo significance were evaluated to determine useful methods, compounds, and kits for treating, reversing, and preventing type 1 diabetes, type 2 diabetes, and insulin resistance, and related vascular complications of inflammation and obesity. Based on the data described, an inhibitor to the 12-LO pathway, and in particular, to 12(S)-HETE and/or 12-HPETE, generated in the 12-LO pathway, would reduce β-cell apoptosis and protect β-cells from inflammatory damage.

In contrast, human pancreatic islets do not express 15-LO in the basal state or after cytokine addition.

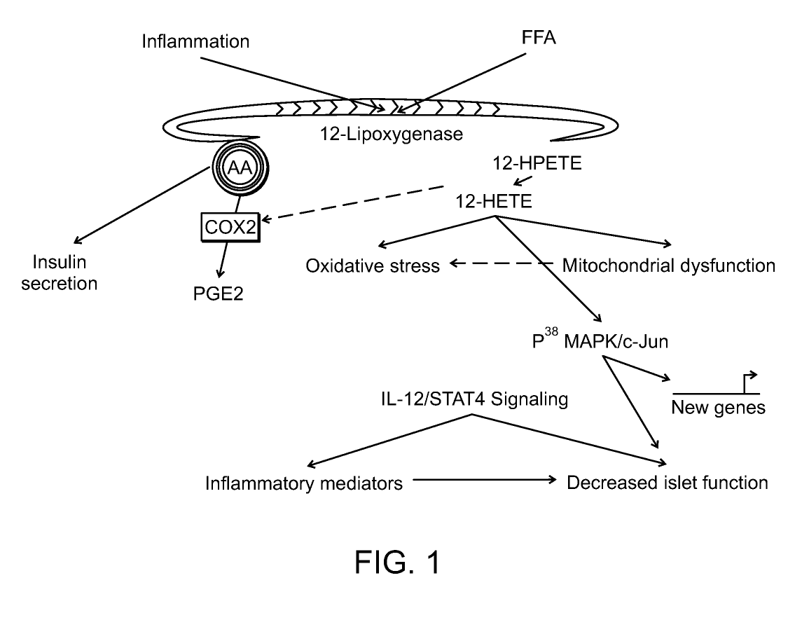

Disclosed are data that show that inflammatory cytokines induce 12-LO RNA and protein expression. Previously, several studies have suggested that diabetes is associated with increased production of 12(S)-HETE (3,8,20). However, the data presented herein documents for the first time the detrimental effects of 12(S)-HETE in human pancreatic islets by inhibiting insulin secretion, reducing metabolic activity, and inducing cell death in human pancreatic islets. Based on this data, it can be concluded that 12(S)-HETE inhibits insulin secretion in human islets at low concentrations that can be produced in vivo. Thus, 12(S)-HETE reduces metabolic activity in human islets, leading to β-cell damage and increased cell death in human islet cells. As shown in FIG. 1, 12-HPETE is a precursor to 12(S)-HETE and can also be targeted.

These effects on human islets may be mediated by the p38-MAPK signaling pathway, which is activated by 12-LO, and repressed in the absence of 12-LO. The disclosure presents evidence which shows that low concentrations of 12(S)-HETE reduces islet function in a manner consistent with p38-MAPK activation. This data supports the hypothesis that 12-LO products act directly on the β-cells in islets. Because 12(S)-HETE can dramatically increase inflammatory cytokine production from macrophages (28), 12(S)-HETE action may also increase local inflammation in islets by the release of cytokines.

Previously it had been shown that MAPKs may participate in cytokine-induced β-cell toxicity (22). Both p38-MAPK kinases and JNK are considered to be important signaling mechanisms in cytokine-induced damage and cell death in islets and β-cells (25). In this disclosure, siRNA technology was used to examine the in vivo role of 12-LO in the p38-MAPK signaling pathway. When 12-LO was specifically knocked down with an siRNA target to that enzyme in C57BL/6J mice, there was a decrease in endogenous pp38-MAPK, whereas the phosphorylated JNK level was not changed. Islets from 12-LO-null mice also showed reduced pp38-MAPK protein activity compared with islets from control C57BL/6 mice but no differences in JNK. Thus, based on the evidence in this disclosure, 12(S)-HETE and cytokines activate p38-MAPK kinases in human islets. In addition, as shown in FIG. 1, p38-MAPK kinase activation in human islets by inflammatory cytokines is blocked by the 12-LO inhibitor, cinnamyl-3,4-dihydroxy-α-cyanocinnamate (CDC), implicating 12(S)-HETE as one mediator of cytokine-induced p38-MAPK kinase activation.

These data show for the first time that 12-LO products inhibit insulin secretion, reduce metabolic activity, and induce cell death in human islets. These data also indicates that human 12-LO activation is an important local pathway for mediating β-cell dysfunction or reduced β-cell mass in diabetes. Development and use of compositions to reduce human 12-LO activity offer an approach to allow functional regeneration and prevent progression of both type 1 and type 2 diabetes to fully developed β-cell destruction.

Thus, provided herein are methods for reducing human 12-LO action using pharmacological or molecular approaches to protect human β-cells from inflammatory injury. One aspect of the invention entails therapy to treat, reverse, or prevent type 1 or type 2 diabetes by reducing 12(S)-HETE production through, for example, inhibition of 12-LO. Development and use of drugs to reduce 12-LO activity offer a novel approach to allow functional regeneration and prevent progression to fully developed β-cell destruction. In further aspects of the invention, the invention entails therapy to treat, reverse, or prevent diabetes or pre-diabetes complications including, for example, insulin resistance, hypertension, atherosclerosis, hypoglycemia, diabetic ketoacidosis, nonketotic hyperosmolar coma, cardiovascular disease, chronic renal failure, and retinal damage. The invention further is directed to identifying compounds that inhibit the 12-LO pathway to reduce the production of HETEs and protect pancreatic β-cells from inflammatory damage and that these compounds are useful for the treatment or prevention of type 1 or type 2 diabetes. The invention is also directed to identifying agents, either small molecule compounds or biologics, that inhibit the 12-LO pathway to reduce the production of HETEs and protect pancreatic β-cells from inflammatory damage.

2. Differential Expression of 12/15 Lipoxygenases in Adipose Tissue

Adipose tissue inflammation is a major factor leading to cardiovascular disease and type 2 diabetes. 12/15 lipoxygenases (12/15 LO or ALOX) play an important role in the generation of inflammatory mediators and downstream immune activation. Moreover, until now, there had been no studies evaluating the expression of various ALOX enzymes in human subcutaneous (sc) or omental (om) adipose tissue in obese humans, nor have there been studies evaluating methods of treating, reversing, or preventing diabetes and/or diseases associated with obesity by inhibiting LO products.

Disclosed is the determination that ALOX15a is selectively expressed only in om tissue. The gene expression and sources of ALOX isoforms and relevant downstream cytokines in sc and om adipose tissue in obese humans show that ALOX isoforms are expressed solely in the stromal vascular fractions (SVF). Gene expression for ALOX15a, ALOX15b, ALOX 12, IL-12a, IL-12b, IL6, IFNγ and CXCL10 was analyzed by real-time PCR in sc and om adipose tissue, adipocytes and SVF. In a paired analysis, all ALOX isoforms, IL6, IL12a and CXCL10 were significantly higher in om versus sc adipose tissue. Moreover, immunohistochemistry revealed selective localization of ALOX15 in the vasculature and in the immune infiltrate in om adipose tissue.

The data also shows for the first time that 12/15-LO play a major role in generating inflammatory mediators that lead to insulin resistance and vascular disease in rodent models of obesity. Thus, inhibiting the 12/15-LO pathway can be used as a method for identifying compounds that are useful for the treatment or prevention of insulin resistance and related vascular complications of inflammation and obesity. The invention is directed to identifying and using compounds that inhibit the 12/15-LO pathway to reduce HETEs reduces inflammatory cytokine production and prevents insulin resistance.

In further aspects of the invention, the invention entails therapy to treat, reduce, reverse, or prevent complications associated with increased visceral obesity and inflammation by inhibiting or reducing 12/15-LO activity. The complications treated by inhibiting 12/15-LO activity includes, for example, diabetes, pre-diabetes, insulin resistance, hypertension, hypoglycemia, diabetic ketoacidosis, atherosclerosis, nonketotic hyperosmolar coma, nonalcoholic steatohepatitis (“NASH”) and related complications of cirrhosis and liver cancer, nerve damage, renal disease, chronic renal failure, retinopathy, metabolic syndrome, and various forms of cancer such as, for example, endometrial cancer, pancreatic cancer, liver cancer, colon cancer, prostate cancer, and breast cancer.

3. Identification of Compounds that Modulate LO Activity

The present invention also provides methods of identifying small molecules or agents which modulate or regulate the 12-LO and 15-LO pathway through any technique or assay known to those of skill in the art that is able to measure the changes to the pathway, typically through high-throughput screening (HTS). With HTS, the target, for example 12(S)-HETE, is tested against large libraries of small molecules for their ability to modify the target. In this illustration, the small molecules or test agents will be screened for their ability to inhibit the 12(S)-HETE activity. Further, HTS can be used to determine the selectivity of the small molecule or agent. The ideal small molecule will interfere with only the chosen target, but not other, related targets. To this end, other screening runs will be made to see whether the “hits” against the chosen target will interfere with other related targets—this is the process of cross-screening. Cross-screening is important, because the more unrelated targets a small molecule hits, the more likely that off-target toxicity will occur with that small molecule. In addition, in identifying small molecules that are useful as inhibitors, kinetic assays can be performed which indicate that the class of inhibitor is tight binding, reversible, and does not reduce the active-site ferric ion of the lipoxygenase. The small molecules or agents may include small organic compounds, small peptides, microRNA, siRNA, hair-pin RNA, and antisense-oligopeptides.

4. Compositions, Methods of Treatment and Kits

The administration of compounds that inhibit or reduce 12-LO activity may be by any suitable means that results in the treatment, reversal, or prevention of diabetes. Moreover, the administration of compounds that inhibit or reduce 12-LO and/or 15-LO activity may be by any suitable means that results in the treatment, reversal, or prevention of insulin resistance and related vascular complications of inflammation and obesity. The 12-LO and 15-LO pathway inhibitors may be contained in any appropriate amount in any suitable carrier substance, and are generally present in amounts totaling 1-95% by weight of the total weight of the composition. The composition may be provided in a dosage form that is suitable for oral, parenteral (e.g., intravenous, intramuscular), rectal, cutaneous, subcutaneous, topical, transdermal, sublingual, nasal, vaginal, intrathecal, epidural, or ocular administration, or by injection, inhalation, or direct contact with the nasal or oral mucosa. Thus, the compositions may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice.

Individually or separately formulated agents can be packaged together as a kit. Non-limiting examples include but are not limited to kits that contain, e.g., two pills, a pill and a powder, a suppository and a liquid in a vial, two topical creams, etc. The kits can include optional components that aid in the administration of the unit dose to patients, such as vials for reconstituting powder forms, syringes for injection, customized IV delivery systems, inhalers, etc. Additionally, the unit dose kits can contain instructions for preparation and administration of the compositions.

The kits may be manufactured as a single use unit dose for one patient, multiple uses for a particular patient (at a constant dose or in which the individual compounds may vary in potency as therapy progresses). Alternatively, the kits may contain multiple doses suitable for administration to multiple patients, such as bulk packaging. The kits components may be assembled in, e.g., cartons, blister packs, bottles, or tubes.

Formulations for oral use include tablets containing the active ingredients in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The compounds can be administered, for example, every four hours, three times daily, twice daily, once daily, once weekly, two to three times weekly, once monthly, twice monthly, or as needed to inhibit or reduce 12-LO or 15-LO activity. Further, the compounds can be administered, for example, by oral, parenteral, intravenous, intramuscular, cutaneous, subcutaneous, topical, transdermal, sublingual, nasal, epidural, or ocular administration, or by injection, inhalation, or direct contact with the nasal or oral mucosa.

### EXAMPLES

The following examples are intended to illustrate, and not limit, the disclosed methods, compounds, and kits.

### Example 1

### Islet Preparation

Human islets, available from the Islet Cell Resource Consortium and the Juvenile Diabetes Research Foundation Basic Science Human Islet Distribution Program, were incubated overnight in Miami medium at 37° C. and 5% CO₂ before experiments. Mouse pancreatic islets were isolated from C57BL/6 mice by collagenase digestion using a previously described protocol (10) that was modified to include Histopaque centrifugation (11). After clamping the common bile duct at the duodenum, the pancreas was perfused through the common bile duct with 5 ml of 1.4 mg/ml collagenase P (Roche Diagnostics, Indianapolis, Ind.) in fresh Hanks' balanced salt solution (HBSS) (Invitrogen, Carlsbad, Calif.) with 1% BSA and 4.2 mM sodium bicarbonate. The perfused pancreas was removed and incubated at 37° C. for 8-11 minutes in 1 ml HBSS solution. After incubation, samples were shaken vigorously by hand. Samples were then washed with HBSS, and the pellet was resuspended. Samples were strained through mesh with 35 linear openings per inch, centrifuged, and resuspended in room temperature Histopaque 1077 (Sigma-Aldrich, St. Louis, Mo.). Equal volumes of HBSS were gently added onto Histopaque layer, resulting in a discontinuous gradient. Samples were centrifuged for 20 minutes at 1200 rpm in Eppendorf Centrifuge 5810R (Eppendorf North America, Hauppauge, N.Y.) at room temperature. Supernatant was washed, and islets were transferred to a petri dish containing RPMI 1640 supplemented with 11 nm glucose (Invitrogen). All islets were incubated overnight to allow sufficient recovery time from collagenase digestion before any experiments were performed.

Human islets were treated with stable compounds derived from LOs: 12(S)-HETE, 15-HETE, 12-HPETE, and 12-RHETE and then examined insulin secretion and islet viability. The p38-MAPK(p38) and JNK stress-activated pathways were investigated as mechanisms of 12-LO-mediated islet inhibition in rodent and human islets.

### Drug Treatments

The cytokine combination chosen as a means of inducing inflammatory responses in islets (12,13,15) was human forms of cytokines (BD Scientific, Franklin Lakes, N.J.) used at the following concentrations: 10 ng/ml for TNF-α, 100 ng/ml for interferon-γ (IFN-γ), and 5 ng/ml for IL-1β in PBS. Lisofylline (LSF) was a generous gift of DiaKine Therapeutics (Charlottesville, Va.). HETEs [12(S)-HETE, 15-HETE, 12-HPETE, and 12-RHETE] and 12-LO inhibitor CDC) were purchased from Biomol (Plymouth Meeting, Pa.).

### Glucose-Stimulated Insulin Secretion

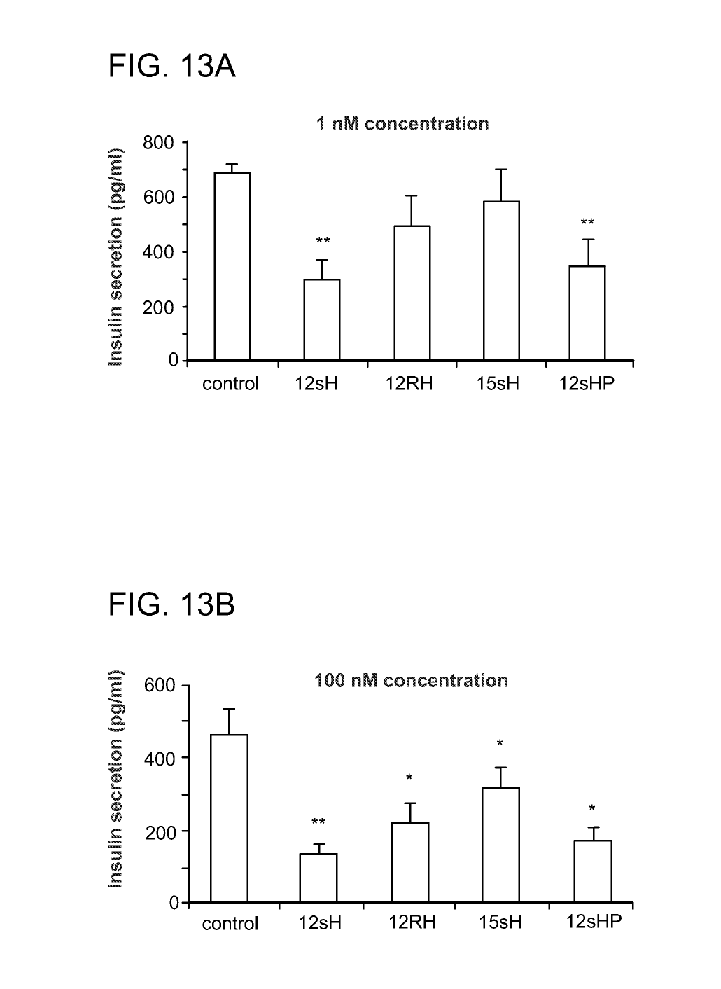

Islets were incubated in a modified Kreb-Ringer buffer with 11 mm glucose after 4-hour HETE exposure, as shown in FIGS. 13A-13C. In other studies, human islets were cultured in CMRL medium-1066 (Invitrogen) with 5% fetal bovine serum overnight and then transitioned to serum free Kreb-Ringer buffer at 37° C., and islets were incubated with and without HETEs in 3 and 18 mM glucose for 4 hours, as shown in FIG. 12. The supernatant was collected after each treatment, and insulin concentration in the supernatant was measured by an enzyme immunoassay method (Mercodia, Uppsala, Sweden) with a mouse or human insulin standard. The intraassay variation was less than 4%, and the interassay variation was less than 10%.

### Cell Death Measurements

Islets were treated with 20 μg/ml of propidium iodide (PI) and incubated for 10 minutes. Islets were imaged once under brightfield illumination to determine the islet borders and imaged again to measure PI fluorescence using 535-nm excitation and 617-nm emission as previously reported (11). Islets were circled, and the mean PI fluorescence intensity was determined for each islet individually. Three separate trials from three donors were conducted.

Small Interfering RNA (siRNA) Studies

Stabilized siRNAs for ip injections were synthesized by Dharmacon (Chicago, Ill.). Groups of 10-wk-old C57BL/6J male mice (Jackson Laboratory, Bar Harbor, Me.) received daily ip injections of 1.6 mg/kg siRNA prepared in 0.9% saline or vehicle alone (0.9% saline) for 3 days as described in (15). For in vitro studies, islets from each group of injected mice were harvested on the fourth day and pooled before analysis. Injections with each siRNA were performed at least three different times. siRNA sequences were as follows: si-Control, 5′-AAAGUCGACCUU-CAGUAAGGA-3′; and si-Alox 15, 5′-GGAUAAGGAAAUU-GAGAUU-3′.

### Western Immunoblotting

Equal amounts of whole islet cell protein extracts were separated on polyacrylamide-sodium dodecyl sulfate gels and transferred to polyvinylidene difluoride transfer membranes (GE Healthcare, Buckinghamshire, UK). The blots were probed with primary antibodies, and bands were detected using horseradish peroxide-conjugated secondary antibodies (GE Healthcare UK Limited, Chalfont, UK) and the enhanced chemiluminescence detection system (GE Healthcare).

### Antibodies

Rabbit PAb anti-p38-MAPK (pTpY180/182) and anti-c-Jun N-terminal kinase (JNK)-1 and -2 (pTpY183/185) were purchased from Alpha BioSource/Invitrogen and were used at a 1:500 dilution. Mouse monoclonal anti-p38-MAPK (A-12) and rabbit polyclonal anti-actin (1-19) were purchased from Santa Cruz Biotechnology (Santa Cruz, Calif.). p38-MAPK was used at a 1:500 dilution, and actin was used at a 1:2500 dilution. Rabbit anti-12-LO (see Ref. 8) was used at a 1:1000 dilution.

### Cell Viability Evaluations

Viability was assessed by detecting 3-(4,5-dimethylthiazol-2-yl)-2,5-diphenyltetrazolium bromide (MTT; Sigma-Aldrich) metabolism as previously described (17).

### Statistics

Statistical analysis was performed using Graph Pad Prism version 4.03 software (GraphPad Software, Inc., San Diego, Calif.). All data are presented as mean±SE, unless otherwise stated. One-way ANOVA followed by a Tukey post test was used for comparing all groups unless otherwise stated. A P value<0.05 was used to indicate statistical significance.

### RNA Extraction and Real-Time PCR

RNA was prepared using the RNeasy Protect Mini Kit (QIAGEN, Gaithersburg, Md.) for human islets. cDNA was made from 5 μg of total RNA using Moloney murine leukemia virus reverse transcriptase (Invitrogen) in 20 μl reaction volume using random hexamers (Invitrogen). For quantitative measurement of PCR products, TaqMan was used (Applied Biosystems Inc., Foster City, Calif.). Three microliters of the cDNA reaction (5-fold diluted) were used as template for PCR in a reaction volume of 25 μl for PCR. Thermal cycling was performed using the iCycler (Bio-Rad, Hercules, Calif.). For the amplification of 12-LO, the cycling conditions were 95° C. for 30 sec, 60° C. for 1 minute. All reactions were performed in triplicate, and the data were normalized to a housekeeping gene, actin, and evaluated using the 2^{-ΔΔCT} method; ALOX12 TaqMan probe was used. Expression levels are presented as fold induction of transcript related to control.

### Selective Effects of LO Products to Inhibit Insulin Secretion

LOs catalyze the oxidation of linoleic acid and arachidonic acid, generating products of varying stability. These include HPETEs, which are subsequently reduced to more stable HETEs by glutathione peroxidase. Referring to FIGS. 13A and 13B, 12(S)-HETE, 15(S)-HETE, 12-HPETE, and 12-RHETE were tested at 1 and 100 nM doses to examine their effects on insulin secretion. Human islets were incubated with the various HETEs for 4 hours and then assessed for insulin secretion during a 1-hour period incubated in 11 mM glucose. Also confirmed were the effects of 12(S)-HETE on basal (3 mM) and stimulated (18 mM) glucose concentration. The 12-LO product 12(S)HPETE (12sHP) and its more stable derivative 12(S)-HETE (12sh) produced the most substantial reduction in insulin secretion (P<0.05; **, P<0.01).

12(S)-HETE Reduces Metabolic Activity and Insulin Secretion: Partial Reversal with LSF

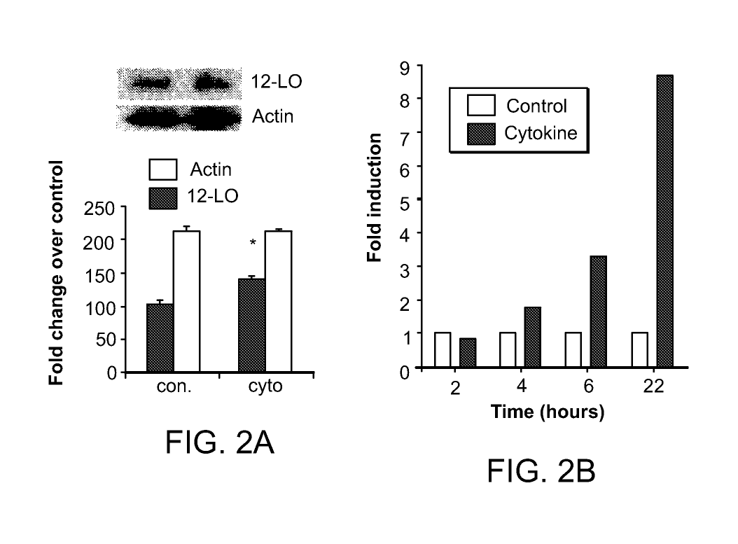

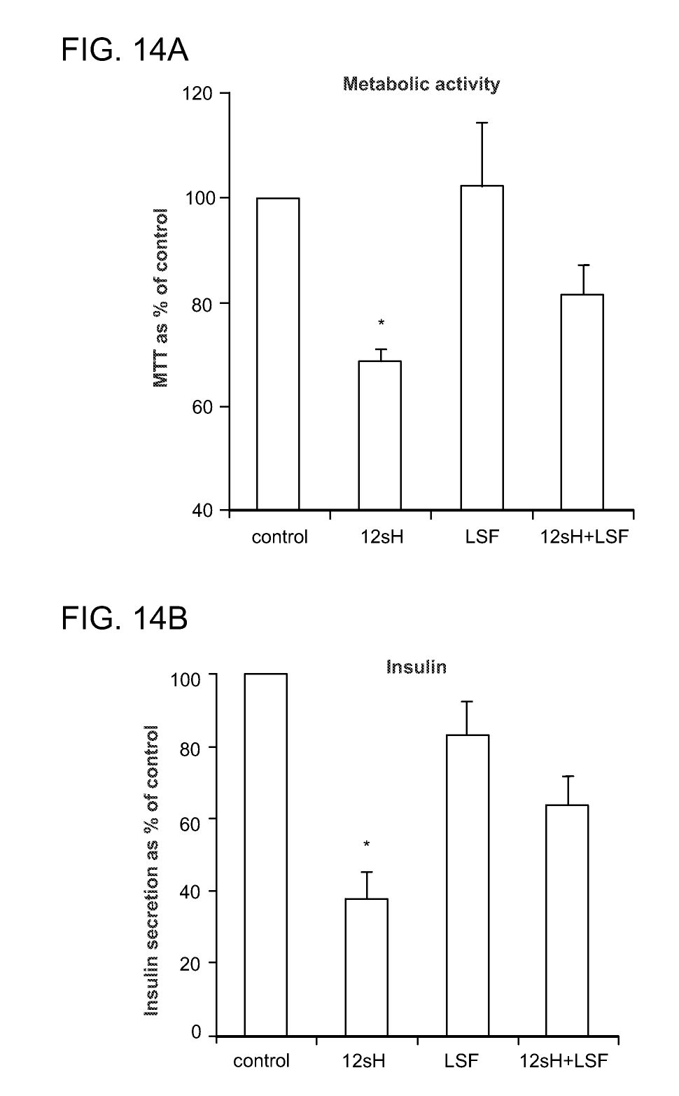

Because the most potent and consistent inhibition of islet function was observed with the 12-LO products, additional experiments were performed on human islets using 12(S)-HETE, the stable form of 12-HPETE. Human islets from four different human donors were pretreated with 50 μm LSF overnight or untreated as indicated. Islets were then treated with 1 nm 12(S)-HETE for 4 hours and incubated in 11 mm glucose for 1 hour to measure islet metabolic activity by MTT assay (A) or insulin secretion (B). As shown in FIG. 2A, the effects of 12(S)-HETE (12sH) on metabolic activity and insulin secretion are partially reversed by the small molecular anti-inflammatory compound LSF as measured by MTT assay. This effect was partially blocked by preincubation with the small molecule anti-inflammatory compound LSF. LSF has previously been shown to protect islet mitochondrial function during the addition of inflammatory cytokines (10). Under the same test conditions, the 12(S)-HETE-induced reduction in insulin secretion was also reversed by LSF treatment (FIG. 14B).

### 12(S)-HETE Induces Cell Death in Human Islets

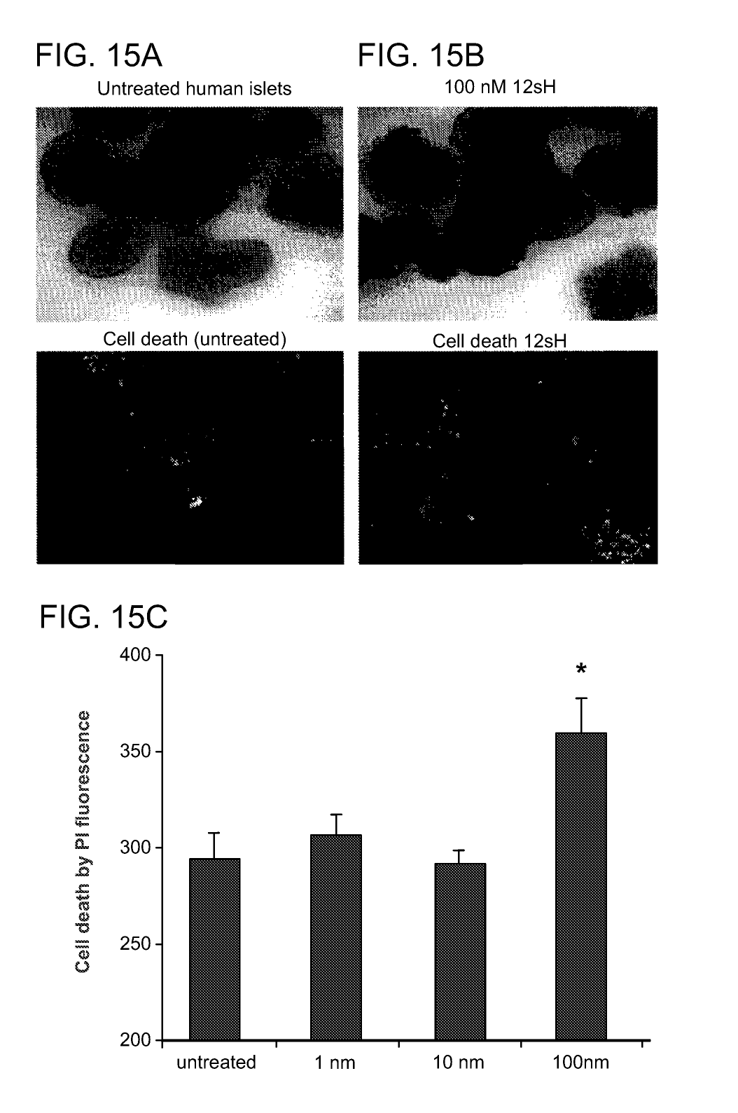

Islet cell death was evaluated by PI staining in islets after overnight incubation with 1, 10, or 100 nM 12(S)-HETE. Whereas human islets showed relatively little PI fluorescence in untreated conditions (FIG. 15A), a significant increase in PI fluorescence was observed among islets incubated overnight with 100 nM 12(S)-HETE (FIG. 15B). Data obtained from a dose-response study is shown in FIG. 15C. A significant increase in cell death was produced by 100 nM 12(S)-HETE, as measured by PI staining intensity, whereas no significant increase in cell death was observed with 1 or 10 nM 12(S)-HETE. This finding is consistent with dose-response measures of cell death previously observed with mouse islets (8).

12(S)-HETE Activates Phosphorylated p38-MAPK (pp38) In Human Islets

To determine possible mechanisms of 12-LO-mediated effects, the stress-mediated pathways linked to β-cell damage were investigated. Treatment of human islets with inflammatory cytokines significantly increased 12-LO protein levels compared with the control untreated condition (FIG. 2A). Human islets were treated with cytokines (10 ng/ml for TNF-α, 100 ng/ml for IFN-γ, and 5 ng/ml for IL-β in PBS) and vehicle for 30 minutes and collected for Western blot (A) and for different time points and collected for real time RT-PCR. Analysis by realtime PCR also showed that 12-LO mRNA expression was induced and that it peaked at 22 hours after cytokine treatment (FIG. 2B). Interestingly, the cytokines did not induce significant expression of 5-LO or 15-lipoxygenase-1 expression (data not shown). 12-LO protein levels after cytokine treatment compared with control, and its quantification was normalized to the actin control. The results are presented as the means±SD. *, P<0.05 compared with the corresponding control. 12-LO mRNA expression was determined by using ALOX12 TaqMan probe by quantitative PCR at indicated time points. The data were normalized to total actin, and fold differences were calculated using the 2^{-ΔCT} method.

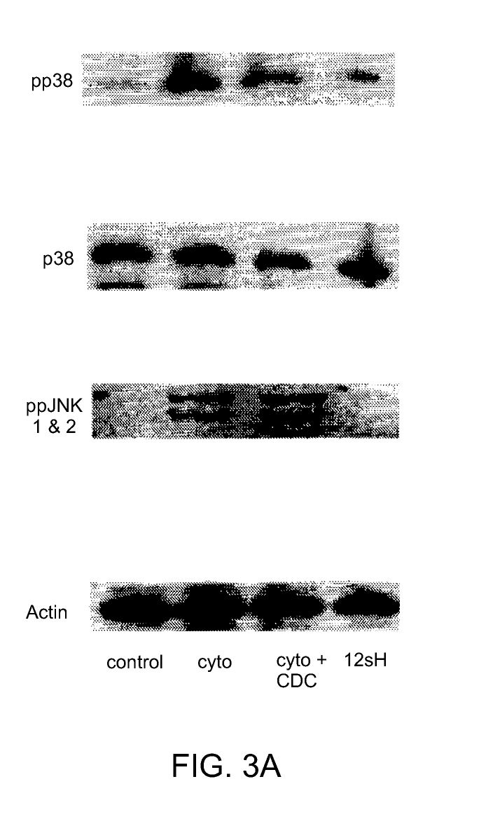

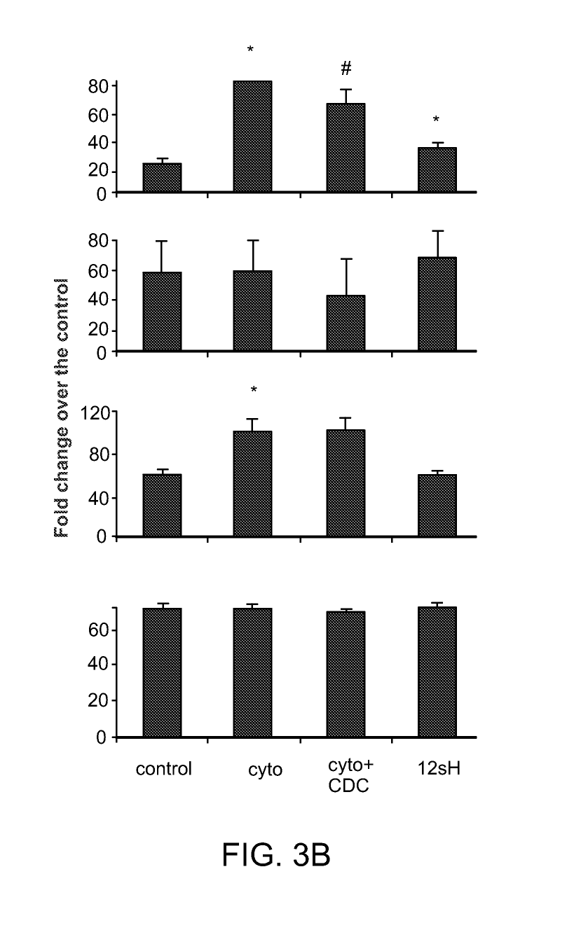

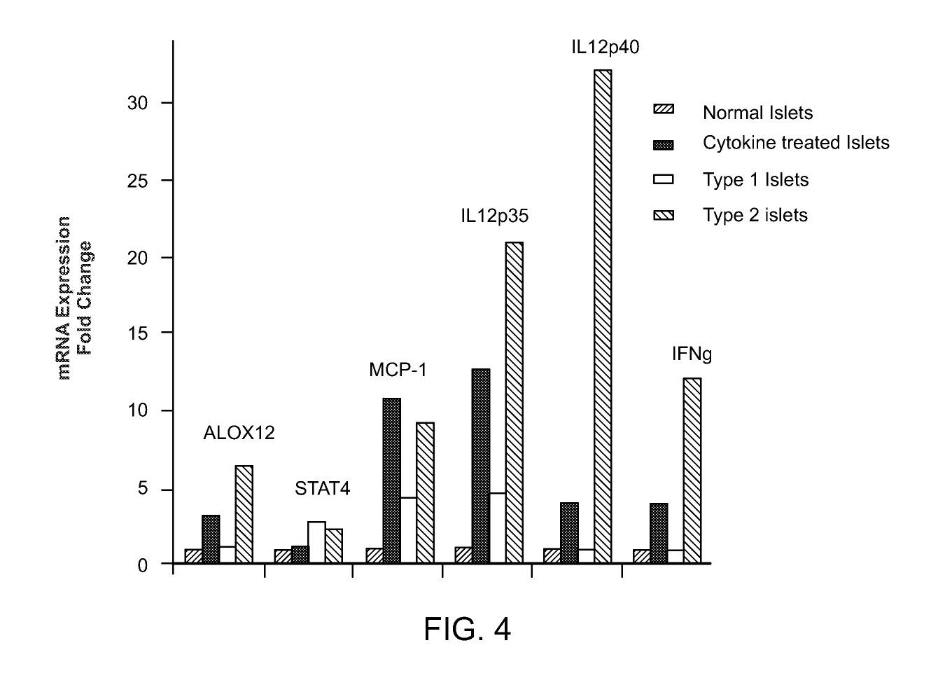

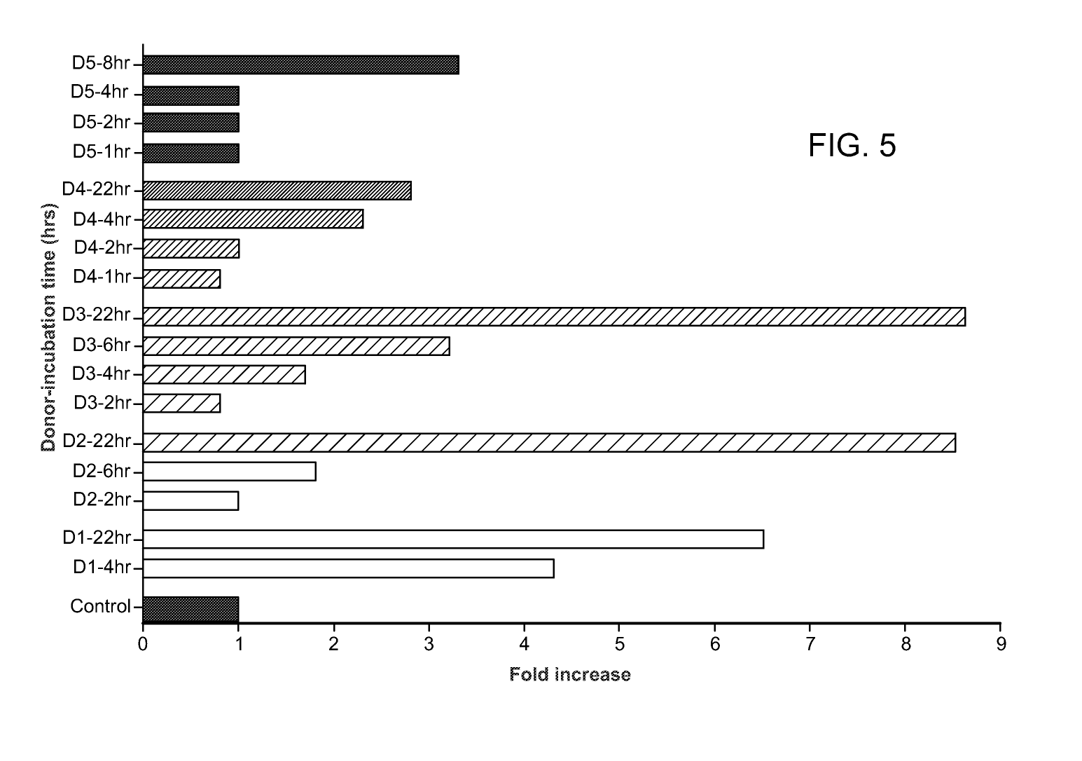

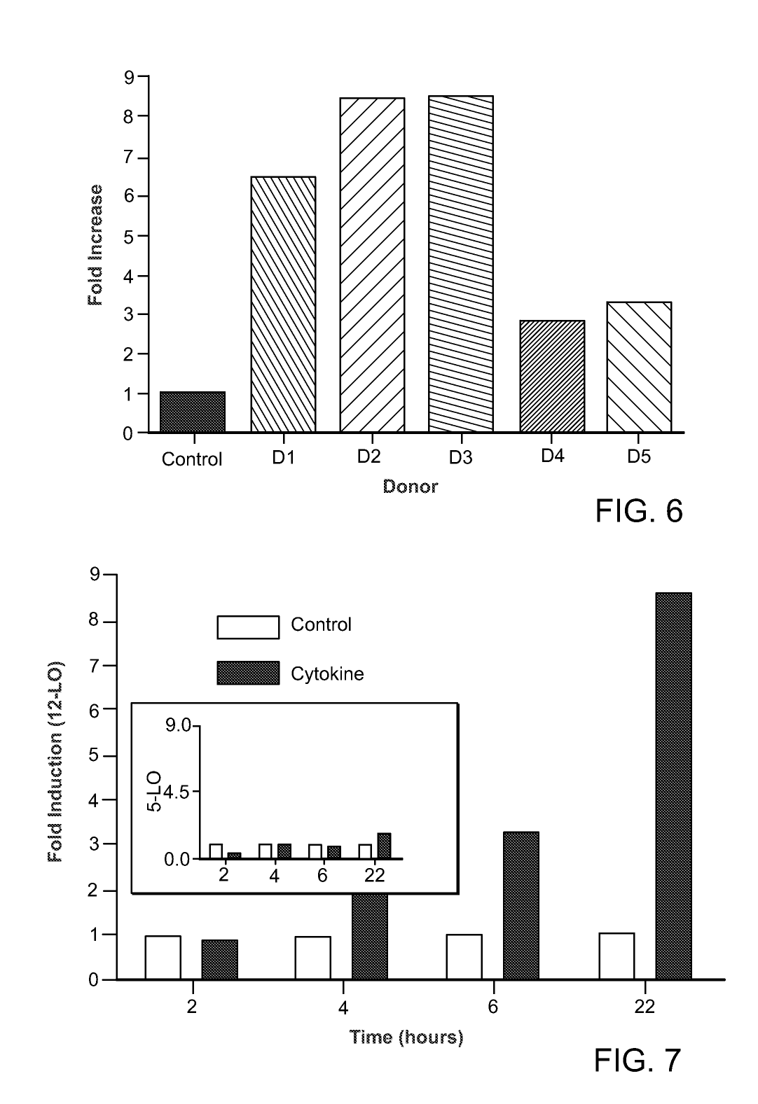

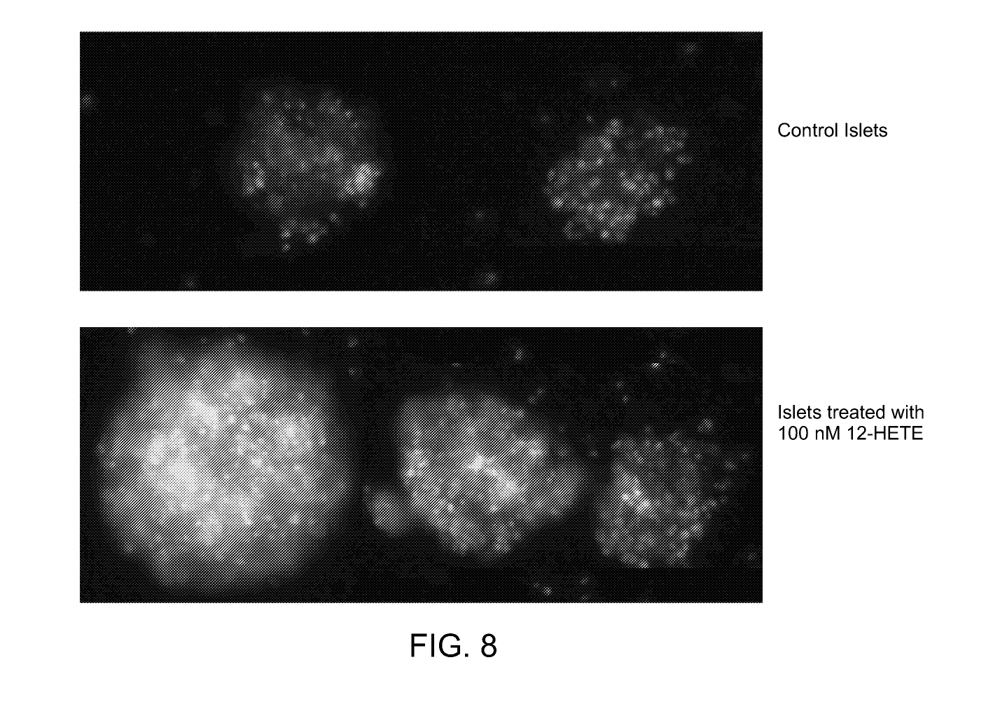

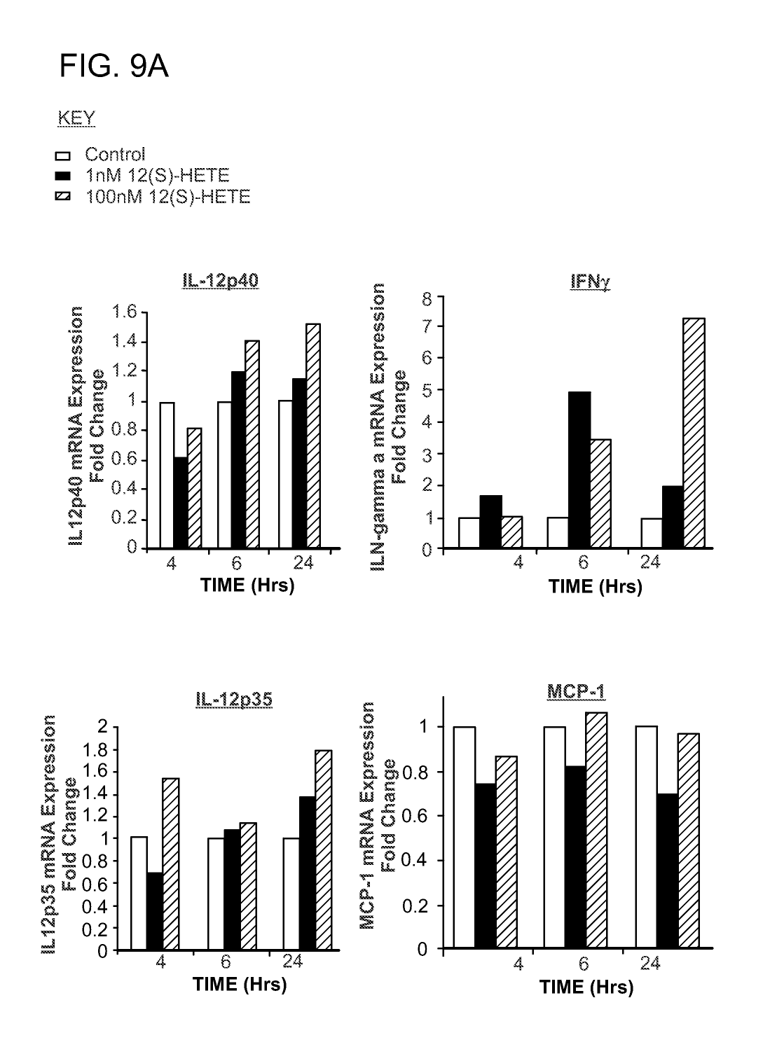

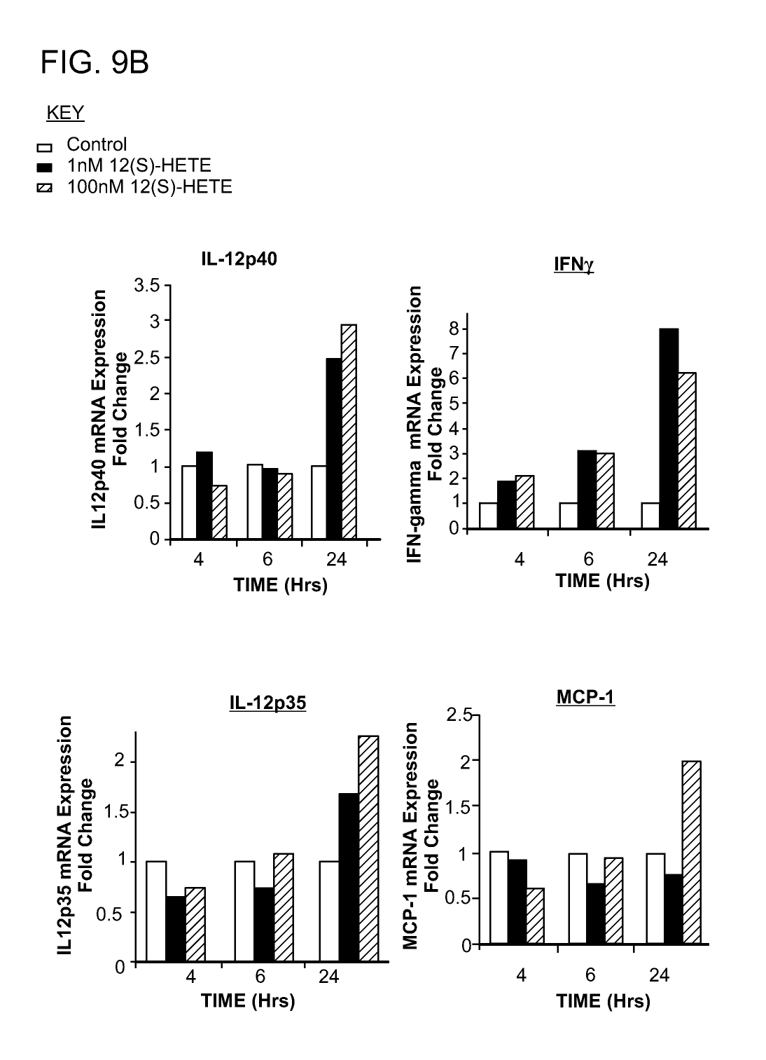

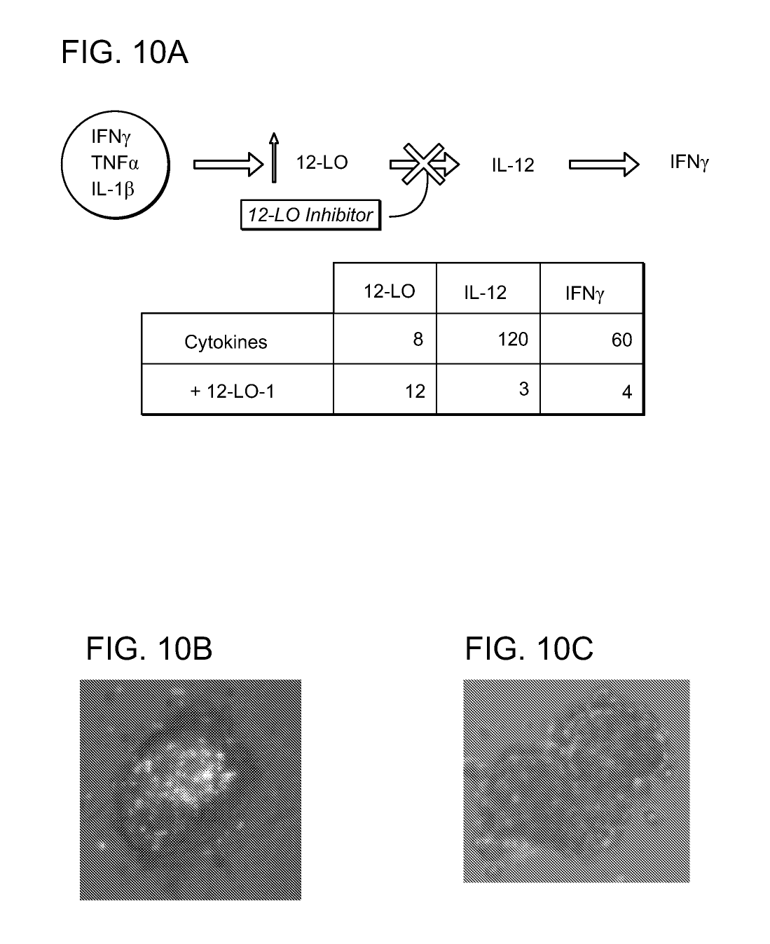

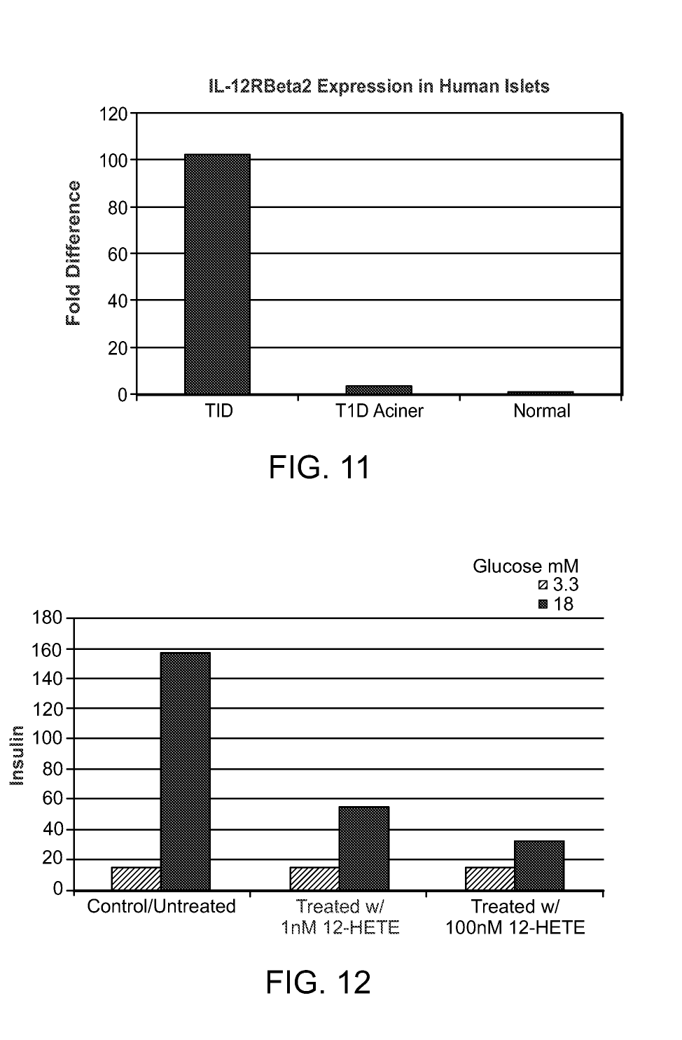

To determine whether treating islets with 12(S)-HETE would affect the activity of two well-known stress-mediated pathways linked to β-cell damage, p38-MAPK and JNK in human islets, were investigated. As shown in FIG. 3A, treatments of cytokines and 12(S)-HETE increased phosphorylated p38 (pp38) without any change in total p38-MAPK (p38). Cytokine-induced pp38-MAPK activity was significantly reduced in islets pretreated with the 12-LO inhibitor CDC. Phosphorylated JNK (ppJNK1 and -2) activity in human islets was increased with cytokine addition but was not affected by 12(S)-HETE, suggesting selectivity of 12-LO products for p38-MAPK activation. The results are quantified in FIG. 3B and show that the 12-LO product 12(S)-HETE could be a mediator of cytokine-induced toxicity in human islets through activation of p38-MAPK signaling. The results are presented as the means±SD. *, P<0.05 compared with the corresponding control; #, P<0.05 compared with the cytokine treatment group.

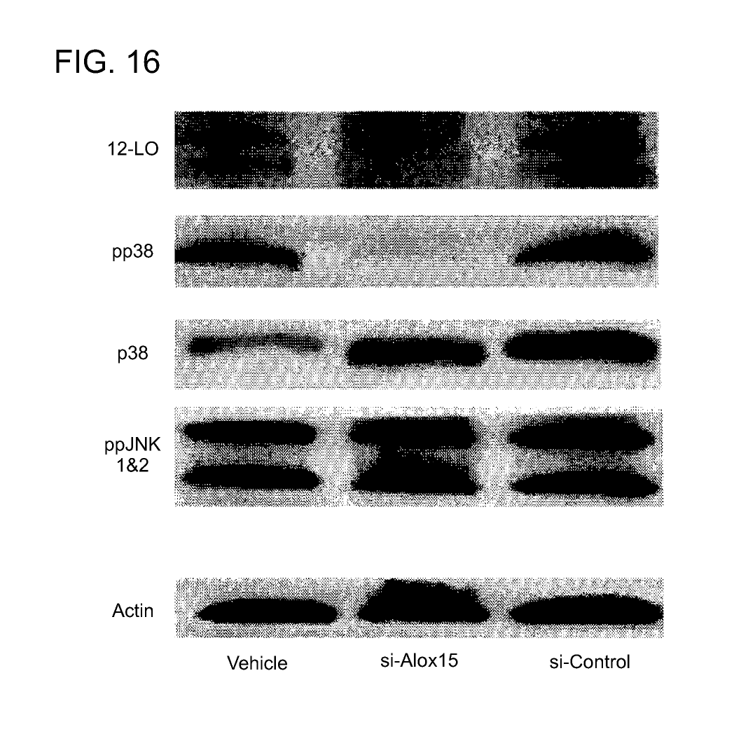

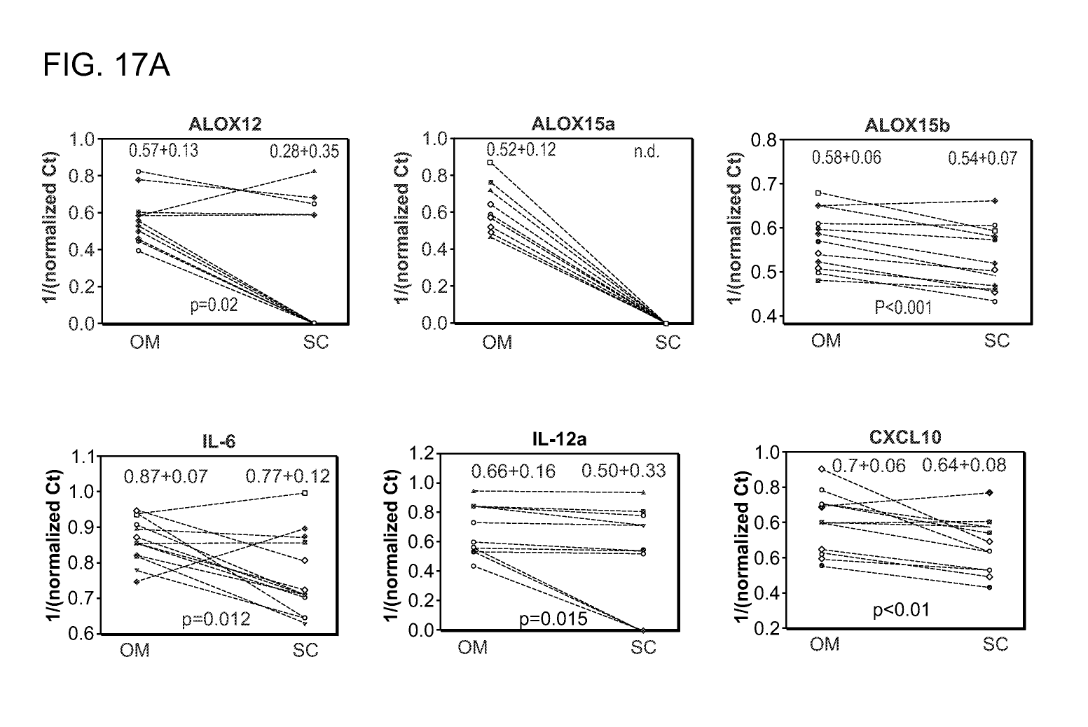

To further evaluate a role of 12-LO in vivo in mediating stress-induced pp38-MAPK in islets, C57BL/6J mice ip were injected with either stabilized siRNA against the mouse form of 12-LO (si-Alox15) or control siRNA (si-Control) daily for 3 days to produce short-term knockdown in 12-LO protein. Islets were isolated from mice that were ip injected on 3 consecutive days with a stabilized siRNA construct against leukocyte 12-LO. As shown in FIG. 16, the siRNA against 12-LO (si-Alox15) considerably reduced 12-LO levels compared with untreated controls (vehicle) or mice injected with missense siRNA (si-Control). Similarly, pp38-MAPK was greatly reduced in the siRNA knockdown group, whereas the total p38-MAPK (p38) expression does not change. Phosphorylated JNK expression (ppJNK1 and -2) was similar in all groups. After 12-LO knockdown, pp38-MAPK activity was significantly reduced. This reduction in p38-MAPK was associated with a similar decrease in 12-LO expression. siRNA knockdown of 12-LO was selective for p38-MAPK because there was no effect on ppJNK1 and -2.

These data suggest that 12(S)-HETE reduces insulin secretion and increases cell death in human islets. The 12-LO pathway is present in human islets, and expression is up-regulated by inflammatory cytokines. Reduction of 12-LO activity thus provides a new therapeutic approach to protect human β-cells from inflammatory injury.

Insulin secretion was consistently reduced by 12(S)-HETE and 12-HPETE. 12(S)—HETE at 1 nM reduced viability activity by 32% measured by MTT assay and increased cell death by 50% at 100 nM in human islets. These effects were partially reversed with LSF. 12(S)-HETE increased phosphorylated p38-MAPK (pp38) protein activity in human islets. Injecting 12-LO siRNA into C57BL/6 mice reduced 12-LO and pp38-MAPK protein levels in mouse islets. The addition of proinflammatory cytokines increased pp38 levels in normal mouse islets but not in siRNA-treated islets.

### Example 2

### Human Subjects and Adipose Tissue Biopsies

To evaluate the implications of ALOX 12 and 15 in pathogenesis of insulin resistance, inflammation in fat, and atherosclerosis, tissue from twelve morbidly obese subjects (3 males and 9 females) qualifying for bariatric surgery were analyzed. The average BMI of the subjects was 42.13±5.94 kg/m² and the average age was 47.8±9.6 years. Subjects were excluded for chronic auto-immune conditions, active tobacco use, type 1 diabetes, active malignancy or infection, or if they were on chronic immunosuppressive or anti-inflammatory medications. Paired samples of sc and om adipose tissue were obtained during each subject's bariatric surgical procedure. Adipose tissue digestion was conducted as described in (42). After filtration, floating adipocytes were collected and the pelleted infranatant contained the SVF.

The procedure for gene expression analysis by real-time PCR has been previously described (42). For real-time PCR, the Taqman probes and primer pairs were from Applied Biosystems. cDNA was amplified in an CFX96 Thermal Cycler (Bio-Rad laboratories, Hercules, Calif.). Results were normalized to 18s RNA or β-actin.

Immunohistochemical staining of adipose tissue was accomplished using tissue biopsies fixed in 10% buffered formalin overnight then embedded in paraffin and incubated for 2 hrs with human anti-ALOX15 antibody (Abnova, 1:100 dilution).

Differences between the sc and om adipose tissue were determined using paired t-tests. Data was represented as mean±SD. The null hypothesis was rejected for a p-value<0.05. Statistical analysis was performed using GraphPad InStat software.

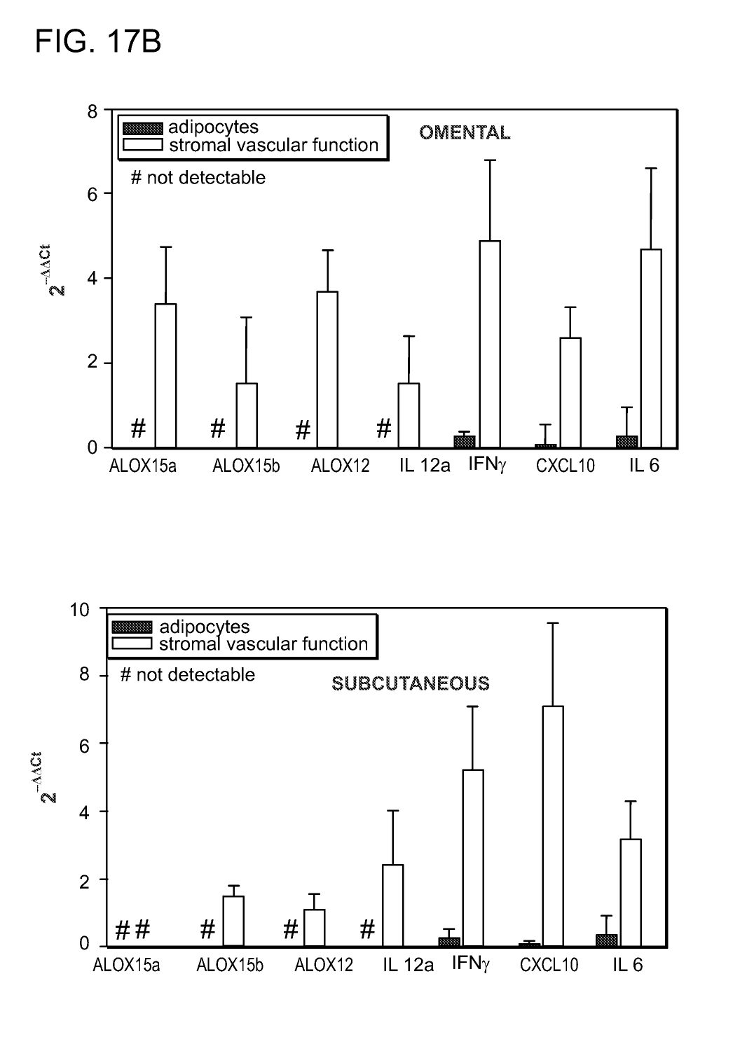

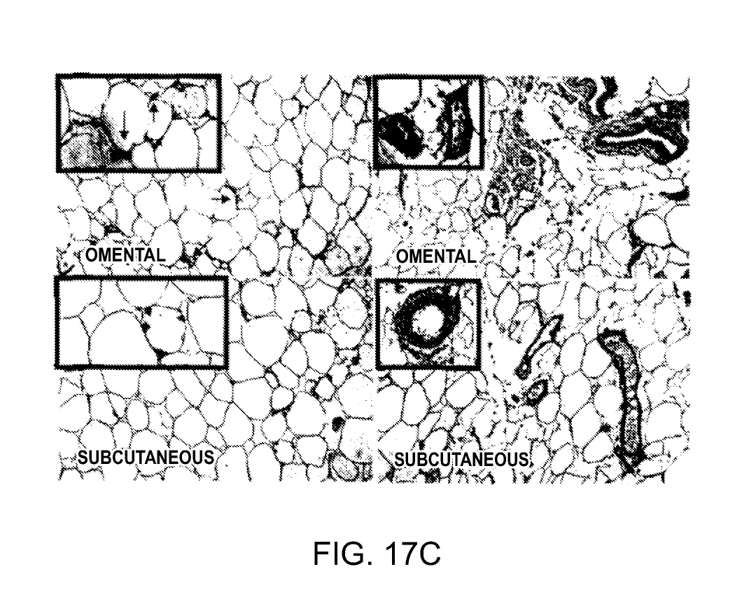

Referring to FIG. 17A, lipoxygenase isoform ALOX15a was expressed only in om tissue, while ALOX15b and ALOX12 were expressed in both sc and om tissue. Significantly higher expression for all of the ALOX isoforms in the om compared to sc adipose tissue was determined using paired analysis. ALOX expression was measured in adipocytes and SVF from sc and om tissue. FIG. 17B shows that all isoforms were solely present in the SVF (FIG. 17B). Further, expression of ALOX15a was determined using immunohistochemistry. Expression was exclusively in the om adipose tissue (FIG. 17C). The strongest expression was detected in the adipose tissue vasculature and to a lesser extent in areas possibly containing immune cells infiltrates.

Inflammatory cytokines such as IL12, IL6, IFNγ and chemokines such as CXCL10 are downstream of 12/15 ALOX activation. Paired analysis of cytokine expression showed significantly higher levels in om versus sc adipose tissue for IL-6, ILI2a and CXCL10 (FIG. 17A). IL12b expression was not detectable in either depot, and no significant difference was measured for IFNγ expression between sc and om adipose tissue. IL-6, IFNγ and CXCL10 were expressed in adipocytes and predominantly in SVF, while IL12a expression was expressed only in SVF (FIG. 17B).

### Histology

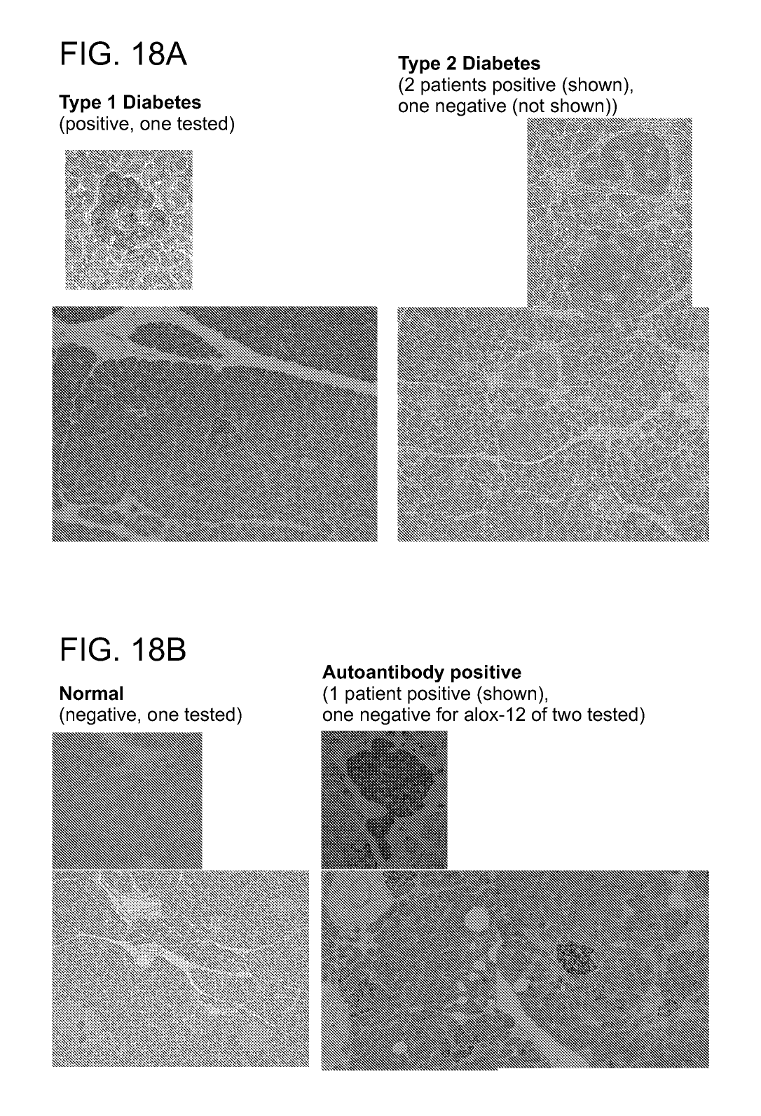

FIG. 18A shows the staining of ALOX 12 in human islet sections from a type 1 diabetic, a type 2 diabetic. FIG. 18B shows the staining of a sample from an auto-antibody positive subject (on the way to developing type 1 diabetes) compared against a control. The human islet sections were stained for ALOX-12, (rabbit poly, Atlas Abs), 1:10 dil, O/N, ImPress antirabbit kit) 10 minutes in substrate (vector VIP “red)).

### Example 3

### Lipoxygenase Expression in Obese Diabetic Human Adipose Tissue and Changes with Bariatric Surgery

The expression of different ALOX isoforms in subcutaneous (sc) and omental (om) human adipose tissue (AT) in obesity and following bariatric surgery was investigated. Expression of the pro-inflammatory cytokines, IL-12, IL-6, and IFNγ were also examined by real-time PCR. Data were analyzed by paired and unpaired Student's t test. Paired biopsies of sc and me adipose tissues were collected from 21 obese subjects, 7 with type 2 diabetes and 14 controls. Body mass index (BMI) was similar between the 2 groups, while hemoglobin Alc (HbAlc) percentage was significantly higher in the type 2 diabetes group. Paired biopsies were also collected from 8 type 2 diabetes subjects at the time of the bariatric surgery and again after a median of 12.5 months. ALOX12, 15a and 15b were expressed solely by the stromal vascular cells in adipose tissue. ALOX12, 15a, 15b, IL-6 AND IL-12a were significantly higher in om vs. sc by 1.3-2.1-fold (p<0.05) in both type 2 diabetes and obese controls. In the om tissue, ALOX12, IL-12a and IFNγ were all significantly higher than type 2 diabetes vs. controls by 1.2-1.8-fold (p<0.05). No differences in expression of any of the genes in sc tissue were found between type 2 diabetes and controls. None of the tested genes correlated with BMI, but ALOX12 showed a significant positive correction (r²=0.573, p<0.05) with HbAlc in the om depot. Following bariatric surgery ALOX12 expression was significantly decreased by 1.6-fold (p<0.05) in om, but not in sc.

It is to be understood that while the methods and procedures have been described in conjunction with the detailed description thereof, the foregoing description is intended to illustrate and not limit the scope of the inventions, which is defined by the scope of the appended claims. Other aspects, advantages, and modifications are within the scope of the following claims. All patent and literature references cited herein are incorporated by reference as if fully set forth in this disclosure.

### REFERENCES

1. Yamamoto S 1992 Mammalian lipoxygenase: molecular structures and functions. Biochem Biophys Acta 1128:117-131
2. Funk C D 1996 The molecular biology of mammalian lipoxygenase and the quest for eicosanoid functions using lipoxygenase-deficient mice. Biochim Biophys Acta 1304:65-84
3. Bleich D, Chen S, Gu J L, Nadler J L1998 The role of 12-lipoxygenase in pancreatic-cells (review). Int J Mol Med 1:265-272
4. McDuffie M, Maybee N A, Keller S R, Stevens B K, Garmey J C, Morris M A, Kropf E, Rival C, Ma K, Carter J D, Tersey S A, Nunemaker C S, Nadler J L 2008 Nonobese diabetic (NOD) mice congenic for a targeted deletion of 12/15-lipoxygenase are protected from autoimmune diabetes. Diabetes 57:199-208
5. Bleich D, Chen S, Zipser B, Sun D, Funk C D, Nadler J L 1999 Resistance to type 1 diabetes induction in 12-lipoxygenase knockout mice. J Clin Invest 103:1431-1436
6. Bleich D, Chen S, Wen Y, Nadler J L 1997 The stress-activated c-Jun protein kinase (JNK) is stimulated by lipoxygenase pathway product 12-HETE in RIN m5F cells. Biochem Biophys Res Commun 230: 448-451
7. Donath M Y, Schumann D M, Faulenbach M, Ellingsgaard H, Perren A, Ehses J A 2008 Islet inflammation in type 2 diabetes: from metabolic stress to therapy. Diabetes Care 31(Suppl 2):S161-S164
8. Chen M, Yang Z D, Smith K M, Carter J D, Nadler J L 2005 Activation of 12-lipoxygenase in proinflammatory cytokine-mediated β-cell toxicity. Diabetologia 48:486-495
9. Cabrera O, Berman D M, Kenyon N S, Ricordi C, Berggren P O, Caicedo A 2006 The unique cytoarchitecture of human pancreatic islets has implications for islet cell function. Proc Natl Acad Sci USA 103:2334-2339
10. Yang Z D, Chen M, Wu R, McDuffie M, Nadler J L 2002 The anti-inflammatory compound lisofylline prevents type I diabetes in non-obese diabetic mice. Diabetologia 45:1307-1314
11. Jahanshahi P, Wu R, Carter J D, Nunemaker C S 2009 Evidence of diminished glucose stimulation and endoplasmic reticulum function in nonoscillatory pancreatic islets. Endocrinology 150:607-615
12. Mandrup-Poulsen T, Bendtzen K, Dinarello C A, Nerup J 1987 Human tumor necrosis factor potentiates human interleukin 1-mediated rat pancreatic β-cell cytotoxicity. J Immunol 139:4077-4082
13. Campbell I L, Iscaro A, Harrison L C 1988 IFN-γ and tumor necrosis factor-a. Cytotoxicity to murine islets of Langerhans. J Immunol 141:2325-2329
14. Cardozo A K, Ortis F, Storling J, Feng Y M, Rasschaert J, Tonnesen M, Van Eylen F, Mandrup-Poulsen T, Herchuelz A, Eizirik D L 2005 Cytokines downregulate the sarcoendoplasmic reticulum pump Ca2+ ATPase 2b and deplete endoplasmic reticulum Ca2+, leading to induction of endoplasmic reticulum stress in pancreatic β-cells. Diabetes 54:452-461
15. Deering T G, Ogihara T, Trace A P, Maier B, Mirmira R G 2009 Methyltransferase Set7/9 maintains transcription and euchromatin structure at islet-enriched genes. Diabetes 58:185-193
16. Yang Z, Chen M, Carter J D, Nunemaker C S, Garmey J C, Kimble S D, Nadler J L 2006 Combined treatment with lisofylline and exendin-4 reverses autoimmune diabetes. Biochem Biophys Res Commun 344:1017-1022
17. Abdel-Rahman E M, Abadir P M, Siragy H M 2008 Regulation of renal 12(S)-hydroxyeicosatetraenoic acid in diabetes by angiotensin AT1 and AT2 receptors. Am J Physiol Regul Integr Comp Physiol 295:R1473-R1478
18. Mandrup-Poulsen T 2001 β-Cell apoptosis: stimuli and signaling. Diabetes 50(Suppl 1):S58-S63
19. Paraskevas S, Aikin R, Maysinger D, Lakey J R, Cavanagh T J, Agapitos D, Wang R, Rosenberg L 2001 Modulation of JNK and p38 stress activated protein kinases in isolated islets of Langerhans: insulin as an autocrine survival signal. Ann Surg 233:124-133
20. Wen Y, Gu J, Vandenhoff G E, Liu X, Nadler J L 2008 Role of 12/15-lipoxygenase in the expression of MCP-1 in mouse macrophages. Am J Physiol Heart Circ Physiol 294:H1933-H1938
21. Nunemaker C S, Chen M, Pei H, Kimble S D, Keller S R, Carter I D, et al. 12-Lipoxygenase-knockout mice are resistant to inflammatory effects of obesity induced by Western diet. Am J Physiol Endocrinol Metab 2008; 295(5):E1065-75
22. Sears D D, Miles P D, Chapman J, Ofrecio J M, Almazan F, Thapar D, et al., 12/15-lipoxygenase is required for the early onset of high fat diet-induced adipose tissue inflammation and insulin resistance in mice. PLoS One 2009; 4(9):e7250
23. Chakrabarti S K, Cole B K, Wen Y, Keller, SR, Nadler J L, 12/15-Lipoxygenase Products Induce Inflammation and Impair Insulin Signaling in 3T3-L1 Adipocytes. Obesity (Silver Spring) 2009
24. Danielsson K N, Rydberg E K, Ingelsten M, Akyurek L M, Jirholt P, Ullstrom C, et al, 15-Lipoxygenase-2 expression in human macrophages induces chemokine secretion and T cell migration. Atherosclerosis 2008; 199(1):34-40
25. O'Rourke R W, Metcalf M D, White A E, Madala A, Winters B R, Maizlin, I I, et al. Depot-specific differences in inflammatory mediators and a role for NK cells and IFN-gamma in inflammation in human adipose tissue. Int J Obes (Lond) 2009; 33(9):978-90
26. Fried S K, Bunkin D A, Greenberg A S, Omental and subcutaneous adipose tissues of obese subjects release interleukin-6: depot difference and regulation by glucocorticoid. J Clin Endocrinol Metab 1998; 83(3):847-50.

1. A method for treating a human patient having or at risk of developing Type I or Type II diabetes, or for treating a human patient receiving an islet graft, the method comprising administering to the patient a therapeutically effective amount of an agent that modulates human arachidonate 12-lipoxygenase activity in pancreatic islet B-cells by reducing the production of 12(S)-HETE or 12-HPETE production.

2. A method for treating a human patient from complications arising from obesity associated with an increase in arachidonate 12-lipoxygenase or arachidonate 15-lipoxygenase activity comprising administering to the patient a therapeutically effective amount of an agent that modulates human 12-lipoxygenase activity or 15-lipoxygenase activity.

3. The method of claim 2, wherein the complications treated are insulin resistance, reduced insulin secretion, hypertension, atherosclerosis, hypoglycemia, diabetic ketoacidosis, nonketotic hyperosmolar coma, cardiovascular disease, chronic renal failure, or retinal damage.

4. The method of claim 2, wherein the agent reduces or inhibits human arachidonate 12-lipoxygenase activity or arachidonate 15-lipoxygenase activity.

5. A method of identifying an agent that can modify human arachidonate 12-lipoxygenase or arachidonate 15-lipoxygenase activity in a human pancreatic islet cell or in human adipose tissue comprising the steps of:
(a) providing a test sample comprising human arachidonate 12-lipoxygenase or arachidonate 15-lipoxygenase or a test sample comprising products of the human arachidonate 12-lipoxygenase or arachidonate 15-lipoxygenase pathway;
(b) contacting the test sample with a test agent;
(c) measuring a change in the response as a result of contacting the test sample with the test agent; and
(d) selecting the test agent that decreases arachidonate 12-lipoxygenase or arachidonate 15-lipoxygenase activity or products of the human arachidonate 12-lipoxygenase or arachidonate 15-lipoxygenase pathway.

6. The method of claim 1, 2, or 5, wherein the agent is a small organic compound, small peptide, microRNA, siRNA, hair-pin RNA, or antisense-oligopeptide.

7. The method of claim 5, wherein the response measured is the expression of 12(S)-HETE, 12-(R)HETE, 12-HPETE, or 12-RHETE.

8. The method of claim 5, wherein the response measured is the expression of ALOX 15a or ALOX 15b.

9. The method of claim 5, wherein the response is induced by contacting the target cell with TNF-α, IFN-γ, IL-1β, IL-6, IL-12, lisofylline, or cinnamyl-3,4-dihydroxy-α-cyanocinnamate.
